(19) United States
(12) Patent Application Publication
(10) Pub. No.: US 2019/0161799 A1
(43) Pub. Date: May 30, 2019

(54) Marker and Method for Determining the Composition or Purity of a Cell Culture and for Determining In Vitro the Identity of a Cartilage Cell or Synovial Cell

(71) Applicant: TETEC Tissue Engineering Technologies AG, Reutlingen (DE)

(72) Inventors: Karin Benz, Göppingen (DE); Christoph Gaissmaier, Kusterdingen (DE)

(21) Appl. No.: 16/321,151

(22) PCT Filed: Jul. 25, 2017
(86) PCT No.: PCT/EP2017/068781

§ 371 Date: Jan. 28, 2019

(30) Foreign Application Priority Data
Jul. 26, 2016 (DE) 10 2016 213 684.4

(51) Int. Cl. C12Q 1/6881; G01N 33/50; G01N 33/68

(52) U.S. Cl. CPC C12Q 1/6881; G01N 33/5005; G01N 33/6887; G01N 2800/10; G01N 2333/4722; G01N 2400/40; C12Q 2600/158

(57) ABSTRACT

The use of a marker for determining the composition or purity of a cell culture, wherein the marker is at least one marker selected from the group consisting of MMP1, ACE, POSTN, SYNPO, SULF1, ARH-GEF28, IGF2BP1, BAIAP2L1, LIMCH1, SCIN, DCLK1, PPL, CRABP2, NES, XPNPEP2 and SLIT3, and the expression level of the at least one marker is determined.

### FIELD OF APPLICATION AND PRIOR ART

The invention relates to markers for determining the composition or purity of a cell culture and for determining in vitro the identity of a chondrocyte or a synovial cell, a method for determining the composition or purity of a cell culture, an in vitro method for determining the identity of a chondrocyte or a synovial cell, the use of a cell culture for producing a medicinal product for novel therapies and the use of a kit for carrying out the aforementioned method.

Increasingly stringent requirements are being placed on the quality of cell-based medicinal products, more particularly cell-based tissue engineering products. This applies in particular with respect to the purity and identity of cells used for producing cell-based medicinal products.

This is a major problem for chondrocyte cultures in particular. Such cultures are frequently prepared using autologous chondrocytes and are used after a specified cultivation period in matrix-associated autologous chondrocyte transplantation.

The particular problem is that the various cell types of mesenchymal origin, such as e.g. osteoblasts, synovial cells and mesenchymal stem cells, do not differ significantly and are therefore practically indistinguishable by means of conventional markers.

In general, endothelial cells, osteoblasts and synovial cells can be considered to be significant in chondrocyte cultures as contaminating cell types. However, synovial cells are particularly critical, as—in contrast to endothelial cells and osteoblasts—they frequently behave identically to chondrocytes under ordinary culturing conditions.

Methods for determining cells and cell cultures are known from EP 2132562 B9.

Nevertheless, there is still a demand for alternative or improved possibilities for carrying out purity controls in cell cultures and for identification of cells.

### OBJECT AND MEANS FOR ACHIEVING OBJECT

An object of the invention is therefore to provide markers that allow purity control of cell cultures, more particularly chondrocyte and/or synovial cell cultures.

Furthermore, an object of the invention is to provide markers that allow identification of chondrocytes and/or synovial cells.

Moreover, an object of the invention is to provide markers that allow discrimination of chondrocytes and synovial cells.

A further object of the invention is to provide corresponding in vitro methods, use of a kit for carrying out said in vitro methods and use of a cell culture for producing a medicinal product for novel therapies.

These objects are achieved according to the invention by use of a marker according to independent claim 1, a method according to claim 13, use of a cell culture according to claim 14, use of a kit according to claim 15 and the applications and methods disclosed in the description. Preferred embodiments are defined in the dependent claims. The wording of all claims is incorporated into the contents of the present description by express reference.

According to a first aspect, the invention relates to the use of a marker for assessing, more particularly for determining the composition or purity of a cell culture.

The marker is characterized in particular by being at least one marker selected from the group consisting of MMP1, ACE, POSTN, SYNPO, SULF1, ARHGEF28, IGF2BP1, BAIAP2L1, LIMCH1, SCIN, DCLK1, PPL, CRABP2, NES, XPNPEP2 and SLIT3, wherein the expression level of the at least one marker is determined.

The term “at least one marker” can refer within the meaning of the present invention to one of the above-mentioned markers or a combination of two or more markers, i.e. a combination of 2, 3, 4, 5, 6, 7, 8, 9, 10, 11, 12, 13, 14, 15 or 16 of the above-mentioned markers or a combination of all of the above-mentioned markers. In particular, the term “at least one marker” within the meaning of the present invention can refer to a ratio of two of the above-mentioned markers.

Furthermore, the term “marker” within the meaning of the present invention can generally refer to a gene product, i.e. the result of expression, such as more particularly the messenger RNA (mRNA) or the protein of one or a plurality of the above-mentioned markers.

The term “expression level” is to be understood within the meaning of the present invention to refer to an expression level on an RNA basis, more particularly a messenger RNA basis (mRNA basis), and/or an expression level on a protein basis.

The term “determine” used in the present invention in connection with the term “expression level” is to be understood within the meaning of the present invention to refer to any biochemical and/or biotechnological method by means of which the expression level of the at least one marker, more particularly on an RNA or protein basis, can be determined, more particularly quantitatively determined.

In proteomic analysis based on mass spectroscopy tests with subsequent bioinformatic evaluation, the inventors succeeded in identifying cartilage cell markers, i.e. chondrocyte markers, and synovial cell markers, i.e. synoviocyte markers, that are suitable for allowing purity control of cell cultures, more particularly chondrocyte and/or synovial cell cultures, and/or identification of the identity of cartilage cells, i.e. chondrocytes, and/or synovial cells, i.e. synoviocytes or synovial fibroblasts. The markers identified in the context of the present invention more particularly allow identification of synovial cell contamination in chondrocyte cultures and/or identification of the presence of chondrocytes in a synovial cell culture and/or discrimination between chondrocytes and synovial cells.

The markers proposed according to the invention can therefore also be referred to as cartilage cell or chondrocyte markers, i.e. as purity or quality markers and/or identity markers for cartilage cells or chondrocytes, and/or as synovial cell or synoviocyte markers, i.e. as purity or quality markers and/or identity markers for synovial cells or synoviocytes.

In a preferred embodiment, the expression level of the at least one marker is determined in a cell sample obtained from the cell culture.

The term “cell sample” is to be understood within the meaning of the present invention to refer to a sample containing cells or composed of cells which is taken from the cell culture.

In a further embodiment, the at least one marker is selected from the group consisting of MMP1, POSTN, SYNPO, SULF1, ARHGEF28, IGF2BP1, BAIAP2L1, LIMCH1, SCIN and combinations of at least two of the aforementioned markers.

In a further embodiment, the at least one marker is selected from the group consisting of ACE, DCLK1, PPL, CRABP2, NES, XPNPEP2, SLIT3 and combinations of at least two of the aforementioned markers.

In a further embodiment, the at least one marker is the protein of MMP1, ACE, POSTN, SYNPO, SULF1, ARHGEF28, IGF2BP1, BAIAP2L1, LIMCH1, SCIN, DCLK1, PPL, CRABP2, NES, XPNPEP2, SLIT3 or a protein combination of at least two of the aforementioned markers.

In particular, the at least one marker can be the protein of MMP1, POSTN, SYNPO, SULF1, ARHGEF28, IGF2BP1, BAIAP2L1, LIMCH1, SCIN or a protein combination of at least two of the aforementioned markers. Alternatively, the at least one marker can be the protein of ACE, DCLK1, PPL, CRABP2, NES, XPNPEP2, SLIT3 or a protein combination of at least two of the aforementioned markers.

In a further embodiment, the at least one marker is an RNA, more particularly the mRNA, of MMP1, ACE, POSTN, SYNPO, SULF1, ARHGEF28, IGF2BP1, BAIAP2L1, LIMCH1, SCIN, DCLK1, PPL, CRABP2, NES, XPNPEP2, SLIT3 or an RNA combination, more particularly an mRNA combination, of at least two of the aforementioned markers.

In particular, the at least one marker can be an RNA, more particularly an mRNA, of MMP1, POSTN, SYNPO, SULF1, ARHGEF28, IGF2BP1, BAIAP2L1, LIMCH1, SCIN or an RNA combination, more particularly an mRNA combination, of at least two of the aforementioned markers. Alternatively, the at least one marker can be an RNA, more particularly the mRNA, of ACE, DCLK1, PPL, CRABP2, NES, XPNPEP2, SLIT3 or an RNA combination, more particularly an mRNA combination, of at least two of the aforementioned markers.

In a further embodiment, the at least one marker is POSTN. As a protein, POSTN is also referred to as “periostin” or “osteoblast specific factor OSF-2.” Periostin is a ligand for alpha-V/beta-3 and alpha-V/beta-5 integrins for supporting the adhesion and migration of epithelial cells.

In particular, the at least one marker can be a combination of POSTN and at least one further marker selected from the group consisting of SYNPO, MMP1, SULF1, ARHGEF28, IGF2BP1, BAIAP2L1, LIMCH1, SCIN, DCLK1, PPL, CRABP2, NES, XPNPEP2, SLIT3 and ACE.

Preferably, the at least one marker is a combination of POSTN and at least one further marker selected from the group consisting of SYNPO, MMP1, SULF1, ARHGEF28, IGF2BP1, BAIAP2L1, LIMCH1 and SCIN.

In a further embodiment, the at least one marker is SYNPO. As a protein, SYNPO is also referred to as “synaptopodin.” Synaptopodin is a cytoplasmic actin-associated protein with a molecular weight of approximately 73 kDa. Synaptopodin occurs both in the podocytes of the kidneys and in the cerebrum.

In particular, the at least one marker can be a combination of SYNPO and at least one further marker selected from the group consisting of POSTN, MMP1, SULF1, ARHGEF28, IGF2BP1, BAIAP2L1, LIMCH1, SCIN, DCLK1, PPL, CRABP2, NES, XPNPEP2, SLIT3 and ACE.

Preferably, the at least one marker is a combination of SYNPO and at least one further marker selected from the group consisting of POSTN, MMP1, SULF1, ARHGEF28, IGF2BP1, BAIAP2L1, LIMCH1 and SCIN.

In a further embodiment, the at least one marker is MMP1. As a protein, MMP1 is also referred to as “matrix metalloproteinase 1”, “interstitial collagenase” or “fibroblast collagenase.”

In particular, the at least one marker can be a combination of MMP1 and at least one further marker selected from the group consisting of ACE, POSTN, SYNPO, SULF1, ARHGEF28, IGF2BP1, BAIAP2L1, LIMCH1, SCIN, DCLK1, PPL, CRABP2, NES, XPNPEP2 and SLIT3. Particularly preferably, the at least one marker is a combination of MMP1 and ACE.

Furthermore, the at least one marker can more particularly be a combination of MMP1 and at least one further marker selected from the group consisting of POSTN, SYNPO, SULF1, ARHGEF28, IGF2BP1, BAIAP2L1, LIMCH1 and SCIN.

In a further embodiment, the at least one marker is SULF1. As a protein, SULF1 is also referred to as “sulfatase 1.” Sulfatase 1 is an enzyme that selectively removes 6-O-sulfate groups from heparin sulfate.

In particular, the at least one marker can be a combination of SULF1 and at least one further marker selected from the group consisting of POSTN, SYNPO, MMP1, ARHGEF28, IGF2BP1, BAIAP2L1, LIMCH1, SCIN, DCLK1, PPL, CRABP2, NES, XPNPEP2, SLIT3 and ACE.

Preferably, the at least one marker is a combination of SULF1 and at least one further marker selected from the group consisting of POSTN, SYNPO, MMP1, ARHGEF28, IGF2BP1, BAIAP2L1, LIMCH1 and SCIN.

In a further embodiment, the at least one marker is ARHGEF28. As a protein, ARHGEF28 is also referred to as “rho guanine nucleotide exchange factor 28.”

In particular, the at least one marker can be a combination of ARHGEF28 and at least one further marker selected from the group consisting of POSTN, SYNPO, MMP1, SULF1, IGF2BP1, BAIAP2L1, LIMCH1, SCIN, DCLK1, PPL, CRABP2, NES, XPNPEP2, SLIT3 and ACE.

Preferably, the at least one marker is a combination of ARHGEF28 and at least one further marker selected from the group consisting of POSTN, SYNPO, MMP1, SULF1, IGF2BP1, BAIAP2L1, LIMCH1 and SCIN.

In a further embodiment, the at least one marker is IGF2BP1. As a protein, IGF2BP1 is also referred to as “insulin-like growth factor 2 mRNA-binding protein 1.”

In particular, the at least one marker can be a combination of IGF2BP1 and at least one further marker selected from the group consisting of POSTN, SYNPO, MMP1, SULF1, ARHGEF28, BAIAP2L1, LIMCH1, SCIN, DCLK1, PPL, CRABP2, NES, XPNPEP2, SLIT3 and ACE.

Preferably, the at least one marker is a combination of IGF2BP1 and at least one further marker selected from the group consisting of POSTN, SYNPO, MMP1, SULF1, ARHGEF28, BAIAP2L1, LIMCH1 and SCIN.

In a further embodiment, the at least one marker is BAIAP2L1. As a protein, BAIAP2L1 is also referred to as “brain-specific angiogenesis inhibitor 1-associated protein 2-like protein 1 (BAI1-associated protein 2-like protein 1).”

In particular, the at least one marker can be a combination of BAIAP2L1 and at least one further marker selected from the group consisting of POSTN, SYNPO, MMP1, SULF1, ARHGEF28, IGF2BP1, LIMCH1, SCIN, DCLK1, PPL, CRABP2, NES, XPNPEP2, SLIT3 and ACE.

Preferably, the at least one marker is a combination of BAIAP2L1 and at least one further marker selected from the group consisting of POSTN, SYNPO, MMP1, SULF1, ARHGEF28, IGF2BP1, LIMCH1 and SCIN.

In a further embodiment, the at least one marker is LIMCH1. As a protein, LIMCH1 is also referred to as “LIM and calponin homology domains 1.”

In particular, the at least one marker can be a combination of LIMCH1 and at least one further marker selected from the group consisting of POSTN, SYNPO, MMP1, SULF1, ARHGEF28, IGF2BP1, BAIAP2L1, SCIN, DCLK1, PPL, CRABP2, NES, XPNPEP2, SLIT3 and ACE.

Preferably, the at least one marker is a combination of LIMCH1 and at least one further marker selected from the group consisting of POSTN, SYNPO, MMP1, SULF1, ARHGEF28, IGF2BP1, BAIAP2L1 and SCIN.

In a further embodiment, the at least one marker is SCIN. As a protein, SCIN is also referred to as “scinderin” or “adseverin.”

In particular, the at least one marker can be a combination of SCIN and at least one further marker selected from the group consisting of POSTN, SYNPO, MMP1, SULF1, ARHGEF28, IGF2BP1, BAIAP2L1, LIMCH1, DCLK1, PPL, CRABP2, NES, XPNPEP2, SLIT3 and ACE.

Preferably, the at least one marker is a combination of SCIN and at least one further marker selected from the group consisting of POSTN, SYNPO, MMP1, SULF1, ARHGEF28, IGF2BP1, BAIAP2L1 and LIMCH1.

In a further embodiment, the at least one marker is DCLK1. As a protein, DCLK1 is also referred to as “doublecortin like kinase-1.”

In particular, the at least one marker can be a combination of DCLK1 and at least one further marker selected from the group consisting of POSTN, SYNPO, MMP1, SULF1, ARHGEF28, IGF2BP1, BAIAP2L1, LIMCH1, SCIN, PPL, CRABP2, NES, XPNPEP2, SLIT3 and ACE.

Preferably, the at least one marker is a combination of DCLK1 and at least one further marker selected from the group consisting of PPL, CRABP2, NES, XPNPEP2, SLIT3 and ACE.

In a further embodiment, the at least one marker is PPL. As a protein, PPL is also referred to as “periplakin.”

In particular, the at least one marker can be a combination of PPL and at least one further marker selected from the group consisting of POSTN, SYNPO, MMP1, SULF1, ARHGEF28, IGF2BP1, BAIAP2L1, LIMCH1, SCIN, DCLK1, CRABP2, NES, XPNPEP2, SLIT3 and ACE.

Preferably, the at least one marker is a combination of PPL and at least one further marker selected from the group consisting of DCLK1, CRABP2, NES, XPNPEP2, SLIT3 and ACE.

In a further embodiment, the at least one marker is CRABP2. As a protein, CRABP2 is also referred to as “cellular retinoic acid-binding protein 2.”

In particular, the at least one marker can be a combination of CRABP2 and at least one further marker selected from the group consisting of POSTN, SYNPO, MMP1, SULF1, ARHGEF28, IGF2BP1, BAIAP2L1, LIMCH1, SCIN, DCLK1, PPL, NES, XPNPEP2, SLIT3 and ACE.

Preferably, the at least one marker is a combination of CRABP2 and at least one further marker selected from the group consisting of DCLK1, PPL, NES, XPNPEP2, SLIT3 and ACE.

In a further embodiment, the at least one marker is NES. As a protein, NES is also referred to as “nestin.”

In particular, the at least one marker can be a combination of NES and at least one further marker selected from the group consisting of POSTN, SYNPO, MMP1, SULF1, ARHGEF28, IGF2BP1, BAIAP2L1, LIMCH1, SCIN, DCLK1, PPL, CRABP2, XPNPEP2, SLIT3 and ACE.

Preferably, the at least one marker is a combination of NES and at least one further marker selected from the group consisting of DCLK1, PPL, CRABP2, XPNPEP2, SLIT3 and ACE.

In a further embodiment, the at least one marker is XPNPEP2. As a protein, XPNPEP2 is also referred to as “X-prolyl aminopeptidase.”

In particular, the at least one marker can be a combination of XPNPEP2 and at least one further marker selected from the group consisting of POSTN, SYNPO, MMP1, SULF1, ARHGEF28, IGF2BP1, BAIAP2L1, LIMCH1, SCIN, DCLK1, PPL, CRABP2, NES, SLIT3 and ACE.

Preferably, the at least one marker is a combination of XPNPEP2 and at least one further marker selected from the group consisting of DCLK1, PPL, CRABP2, NES, SLIT3 and ACE.

In a further embodiment, the at least one marker is SLIT3. As a protein, SLIT3 is also referred to as “slit guidance ligand 3.”

In particular, the at least one marker can be a combination of SLIT3 and at least one further marker selected from the group consisting of POSTN, SYNPO, MMP1, SULF1, ARHGEF28, IGF2BP1, BAIAP2L1, LIMCH1, SCIN, DCLK1, PPL, CRABP2, NES, XPNPEP2 and ACE.

Preferably, the at least one marker is a combination of SLIT3 and at least one further marker selected from the group consisting of DCLK1, PPL, CRABP2, NES, XPNPEP2 and ACE.

In a further embodiment, the at least one marker is ACE. As a protein, ACE is also referred to as “angiotensin-converting enzyme.”

In particular, the at least one marker can be a combination of ACE and at least one further marker selected from the group consisting of MMP1, POSTN, SYNPO, SULF1, ARHGEF28, IGF2BP1, BAIAP2L1, LIMCH1, SCIN, DCLK1, PPL, CRABP2, NES, XPNPEP2 and SLIT3. Particularly preferably, the at least one marker is a combination of ACE and MMP1.

Furthermore, the at least one marker can more particularly be a combination of ACE and at least one further marker selected from the group consisting of DCLK1, PPL, CRABP2, NES, XPNPEP2 and SLIT3.

In a further embodiment, the cell culture is a cartilage cell culture or chondrocyte culture.

The term “cartilage cell culture” or “chondrocyte culture” is to be understood within the meaning of the present invention to refer to a culture that is produced or producible from cartilage tissue, preferably from a cartilage biopsy specimen, more particularly a cartilage/bone punch biopsy specimen, by means of enzymatic digestion and subsequent cultivation of the cells obtained after digestion.

The term “cartilage tissue” can refer within the meaning of the present invention to healthy cartilage tissue, diseased or pathologically modified cartilage tissue or a cartilage tissue regenerate, i.e. a newly created cartilage tissue, preferably as a result of medical treatment, more particularly autologous chondrocyte transplantation (ACT) or matrix-associated autologous chondrocyte transplantation (MACT).

The term “cartilage biopsy specimen” can refer within the meaning of the present invention to a biopsy specimen that contains healthy cartilage tissue, diseased or pathologically modified cartilage tissue or a cartilage tissue regenerate or consists of one of the aforementioned tissues.

The term “cartilage/bone punch biopsy specimen” is to be understood within the meaning of the present invention to refer to a biopsy specimen that contains healthy bone and cartilage tissue, diseased bone and cartilage tissue or pathologically modified bone and cartilage tissue or a bone and cartilage tissue regenerate or consists of one of the aforementioned tissues.

In a further embodiment, the cell culture is a synovial cell culture or synoviocyte culture. The term “synovial cell culture” or “synoviocyte culture” is to be understood within the meaning of the present invention to refer to a culture that is produced or producible from synovial tissue by means of enzymatic digestion and subsequent cultivation of the cells obtained after digestion.

In a further embodiment, the cell culture contains cells that originate from a cartilage biopsy specimen. In particular, the cell culture can contain cells that originate from a cartilage/bone punch biopsy specimen, more particularly a cartilage tissue fragment thereof.

The biopsy specimen, more particularly the cartilage biopsy specimen or cartilage/bone punch biopsy specimen, is preferably a biopsy specimen from a joint, more particularly a knee joint.

In a further embodiment, the cell culture contains cells that originate from a synovial tissue biopsy specimen.

In a further embodiment, the at least one marker is selected from the group consisting of MMP1, POSTN, SYNPO, SULF1, ARHGEF28, IGF2BP1, BAIAP2L1, LIMCH1, SCIN and combinations of at least two of the aforementioned markers, wherein an expression level of the at least one marker above a defined threshold value indicates that the cell culture contains cartilage cells or chondrocytes. The threshold value is preferably greater than or equal to the expression level of the at least one marker in a pure synovial cell culture. The term “pure synovial cell culture” is to be understood within the meaning of the present invention to refer to a synovial cell culture that contains cells exclusively in the form of synovial cells or synoviocytes. The markers mentioned in this paragraph can therefore also be referred to within the meaning of the present invention as cartilage cell markers or chondrocyte markers.

In a further embodiment, the at least one marker is selected from the group consisting of ACE, DCLK1, PPL, CRABP2, NES, XPNPEP2, SLIT3 and combinations of at least two of the aforementioned markers, wherein an expression level of the at least one marker above a defined threshold value indicates that the cell culture contains synovial cells or synoviocytes. The threshold value is preferably greater than or equal to the expression level of the at least one marker in a pure chondrocyte culture. The term “pure chondrocyte culture” is to be understood within the meaning of the present invention to refer to a chondrocyte culture that contains cells exclusively in the form of cartilage cells or chondrocytes. The markers mentioned in this paragraph can therefore also be referred to within the meaning of the present invention as synovial cell markers or synoviocyte markers.

In a further embodiment, the at least one marker is the ratio of ACE to MMP1 (ACE/MMP1 ratio) or the ratio of MMP1 to ACE (MMP1/ACE ratio). In other words, the marker for determining the composition or purity of a cell culture according to a further embodiment is the ratio of ACE to MMP1 or the ratio of MMP1 to ACE. Preferably, for determining the composition or purity of the cell culture, the ratio of ACE to MMP1 is formed, i.e. the ACE/MMP1 ratio is used as a marker for determining the composition or purity of a cell culture. Preferably, a value of the ACE/MMP1 ratio above a defined threshold value indicates that the cell culture contains synovial cells (synoviocytes). The threshold value is preferably less than or equal to the value of the ratio of ACE to MMP1 in a pure synovial cell culture. The expression level of the at least one marker is determined in a further embodiment on an RNA basis, and preferably a messenger RNA basis (mRNA basis).

In a further embodiment, the expression level of the at least one marker is determined on a protein basis.

In a further embodiment, the expression level of the at least one marker is determined by means of a polymerase chain reaction such as the reverse transcriptase polymerase chain reaction or the quantitative real time polymerase chain reaction, an electrophoretic assay, a gel electrophoretic assay such as southern blot, northern blot or western blot, protein chips, antibodies, an immunoassay such as ELISA, a LUMINEX immunoassay or ELISpot assay, an immunofluorescence assay, an immunohistochemical assay, a radioimmunological assay, proteomic analysis, a chromatography-based assay such as HPLC or gel chromatography, a mass spectrographic assay or a flow cytometric assay.

According to a second aspect, the invention relates to the use of a marker for determining in vitro the identity of a cartilage cell (chondrocyte) and/or a synovial cell (synoviocyte), more particularly for discriminating (differentiating) between a chondrocyte and a synovial cell.

The marker is at least one marker that is selected from the group consisting of MMP1, ACE, POSTN, SYNPO, SULF1, ARHGEF28, IGF2BP1, BAIAP2L1, LIMCH1, SCIN, DCLK1, PPL, CRABP2, NES, XPNPEP2 and SLIT3, wherein the expression level of the at least one marker is determined in a cell sample obtained from a cell culture.

In a preferred embodiment, the at least one marker is selected from the group consisting of MMP1, POSTN, SYNPO, SULF1, ARHGEF28, IGF2BP1, BAIAP2L1, LIMCH1, SCIN and combinations of at least two of the aforementioned markers, wherein an expression level of the at least one marker above a defined threshold level indicates that the cell is a cartilage cell or a chondrocyte. The threshold value is preferably greater than or equal to the expression level of the at least one marker in a synovial cell or a synoviocyte.

In a further embodiment, the at least one marker is selected from the group consisting of ACE, DCLK1, PPL, CRABP2, NES, XPNPEP2, SLIT3 and combinations of at least two of the aforementioned markers, wherein an expression level of the at least one marker above a defined threshold level indicates that the cell is a synovial cell or a synoviocyte. The threshold value is preferably greater than or equal to the expression level of the at least one marker in a cartilage cell or in a chondrocyte.

In a further embodiment, the cell culture contains cells that originate from a cartilage biopsy specimen. In particular, the cell culture can contain cells that originate from a cartilage/bone punch biopsy specimen, more particularly a cartilage tissue fragment thereof.

The biopsy specimen, more particularly the cartilage biopsy specimen or cartilage/bone punch biopsy specimen, is preferably a biopsy specimen from a joint, more particularly a knee joint.

In a further embodiment, the cell culture contains cells that originate from a synovial tissue biopsy specimen.

In a further embodiment, the at least one marker is the ratio of ACE to MMP 1 (ACE/MMP1 ratio) or the ratio of MMP1 to ACE (MMP1/ACE ratio). In other words, the marker for in vitro determination of the identity of a cartilage and/or synovial cell according to a further embodiment is the ratio of ACE to MMP1 or the ratio of MMP1 to ACE. Preferably, the at least one marker is the ratio of ACE to MMP 1. In other words, it is preferred according to the invention if the marker for in vitro determination of the identity of a chondrocyte and/or synovial cell is the ratio of ACE to MMP 1. A value of the ACE/MMP1 ratio above a defined threshold value indicates that the cell is a synovial cell. The threshold value is preferably less than or equal to the value of the ratio of ACE to MMP1 in a synovial cell.

With respect to further features and advantages of the use, more particularly of the at least one marker, in order to avoid repetitions, the explanations made in the context of the above description are incorporated herein by reference in their entirety. The embodiments described therein also apply (mutatis mutandis) to the use of the marker according to the second aspect of the invention.

According to a third aspect, the invention relates to a method for assessing, more particularly for determining, the composition or purity of a cell culture.

The method comprises the following steps:
a) obtaining a cell sample from a cell culture,
b) determining the expression level of at least one marker in the cell sample and
c) determining the composition or purity of the cell culture based on the expression level of the at least one marker.

The method is characterized in particular in that the at least one marker is selected from the group consisting of MMP1, ACE, POSTN, SYNPO, SULF1, ARHGEF28, IGF2BP1, BAIAP2L1, LIMCH1, SCIN, DCLK1, PPL, CRABP2, NES, XPNPEP2, SLIT3 and combinations of at least two of the aforementioned markers.

In a further embodiment, the cell culture contains cells that originate from a cartilage biopsy specimen. In particular, the cell culture can contain cells that originate from a cartilage/bone punch biopsy specimen, more particularly a cartilage tissue fragment thereof.

The biopsy specimen, more particularly the cartilage biopsy specimen or cartilage/bone punch biopsy specimen, is preferably a biopsy specimen from a joint, more particularly a knee joint.

In a further embodiment, the cell culture contains cells that originate from a synovial tissue biopsy specimen.

In a further embodiment, the at least one marker is the ratio of ACE to MMP1 (ACE/MMP1 ratio) or the ratio of MMP1 to ACE (MMP1/ACE ratio). Preferably, the at least one marker is the ratio of ACE to MMP1. Preferably, a value of the ratio of ACE to MMP1 above a defined threshold value indicates that the cell culture contains synovial cells (synoviocytes). The threshold value is preferably less than or equal to the value of the ratio of ACE to MMP1 in a pure synovial cell culture.

With respect to further features and advantages of the method, more particularly of the at least one marker, in order to avoid repetitions, the explanations made in the context of the above description, more particularly with respect to the first aspect of the invention, are also incorporated herein by reference in their entirety. The embodiments described therein also apply (mutatis mutandis) to the method according to the third aspect of the invention.

According to a fourth aspect, the invention relates to an in vitro method for determining the identity of a cartilage cell (chondrocyte) and/or a synovial cell (synoviocyte), more particularly for discriminating (differentiating) between a chondrocyte and a synovial cell.

The method comprises the following steps:
a) determining the expression level of at least one marker in a cell sample obtained from a cell culture and
b) determining the identity of the cell based on the expression level of the at least one marker.

The method is characterized in particular in that the at least one marker is selected from the group consisting of MMP1, ACE, POSTN, SYNPO, SULF1, ARHGEF28, IGF2BP1, BAIAP2L1, LIMCH1, SCIN, DCLK1, PPL, CRABP2, NES, XPNPEP2, SLIT3 and combinations of at least two of the aforementioned markers.

In a further embodiment, the cell culture contains cells that originate from a cartilage biopsy specimen. In particular, the cell culture can contain cells that originate from a cartilage/bone punch biopsy specimen, more particularly a cartilage tissue fragment thereof.

The biopsy specimen, more particularly the cartilage biopsy specimen or cartilage/bone punch biopsy specimen, is preferably a biopsy specimen from a joint, more particularly a knee joint.

In a further embodiment, the cell culture contains cells that originate from a synovial tissue biopsy specimen.

In a further embodiment, the at least one marker is the ratio of ACE to MMP 1 (ACE/MMP1 ratio) or the ratio of MMP1 to ACE (MMP1/ACE ratio). Preferably, the at least one marker is the ratio of ACE to MMP1. Preferably, a value of the ratio of ACE to MMP 1 above a defined threshold value indicates that the cell is a synovial cell. The threshold value is preferably less than or equal to the value of the ratio of ACE to MMP1 in a synovial cell.

With respect to further features and advantages of the method, more particularly of the at least one marker, the explanations made in the context of the above description, more particularly with respect to the first and second aspects of the invention, are also incorporated herein by reference in their entirety. The embodiments described therein also apply (mutatis mutandis) to the method according to the fourth aspect of the invention.

According to a fifth aspect, the invention relates to a marker for use in the assessment, more particularly the determination, of the composition or purity of a cell culture.

The marker is at least one marker that is selected from the group consisting of MMP1, ACE, POSTN, SYNPO, SULF1, ARHGEF28, IGF2BP1, BAIAP2L1, LIMCH1, SCIN, DCLK1, PPL, CRABP2, NES, XPNPEP2 and SLIT3, wherein the expression level of the at least one marker is determined.

In a further embodiment, the cell culture contains cells that originate from a cartilage biopsy specimen. In particular, the cell culture can contain cells that originate from a cartilage/bone punch biopsy specimen, more particularly a cartilage tissue fragment thereof.

The biopsy specimen, more particularly the cartilage biopsy specimen or cartilage/bone punch biopsy specimen, is preferably a biopsy specimen from a joint, more particularly a knee joint.

In a further embodiment, the cell culture contains cells that originate from a synovial tissue biopsy specimen.

In a further embodiment, the at least one marker is the ratio of ACE to MMP 1 (ACE/MMP1 ratio) or the ratio of MMP1 to ACE (MMP1/ACE ratio). In other words, the marker for use in assessing composition or purity of a cell culture according to a further embodiment is the ratio of ACE to MMP1 (ACE/MMP1 ratio) or the ratio of MMP1 to ACE (MMP1/ACE ratio).

Preferably, the at least one marker is the ratio of ACE to MMP1. In other words, it is preferred according to the invention if the marker for use in assessing the composition or purity of a cell culture is the ratio of ACE to MMP1. Preferably, a value of the ratio of ACE to MMP1 above a defined threshold value indicates that the cell culture contains synovial cells (synoviocytes). The threshold value is preferably less than or equal to the value of the ratio of ACE to MMP1 in a pure synovial cell culture.

With respect to further features and advantages of the marker, in order to avoid repetitions, the explanations made in the context of the above description, more particularly the first aspect of the invention, are also incorporated herein by reference in their entirety. The embodiments described therein also apply (mutatis mutandis) to the marker according to the fifth aspect of the invention.

According to a sixth aspect, the invention relates to a marker for use in determining in vitro the identity of a cartilage cell (chondrocyte) and/or a synovial cell (synoviocyte), more particularly for use in in vitro discrimination (differentiation) between a chondrocyte and a synovial cell.

The marker is at least one marker that is selected from the group consisting of MMP1, ACE, POSTN, SYNPO, SULF1, ARHGEF28, IGF2BP1, BAIAP2L1, LIMCH1, SCIN, DCLK1, PPL, CRABP2, NES, XPNPEP2 and SLIT3, wherein the expression level of the at least one marker is determined in a cell sample obtained from a cell culture.

In a further embodiment, the cell culture contains cells that originate from a cartilage biopsy specimen. In particular, the cell culture can contain cells that originate from a cartilage/bone punch biopsy specimen, more particularly a cartilage tissue fragment thereof.

The biopsy specimen, more particularly the cartilage biopsy specimen or cartilage/bone punch biopsy specimen, is preferably a biopsy specimen from a joint, more particularly a knee joint.

In a further embodiment, the cell culture contains cells that originate from a synovial tissue biopsy specimen.

In a further embodiment, the at least one marker is the ratio of ACE to MMP1 (ACE/MMP1 ratio) or the ratio of MMP1 to ACE (MMP1/ACE ratio). In other words, the marker for use in determining in vitro the identity of a chondrocyte and/or synovial cell according to a further embodiment is the ratio of ACE to MMP1 (ACE/MMP1 ratio) or the ratio of MMP1 to ACE (MMP1/ACE ratio).

Preferably, the at least one marker is the ratio of ACE to MMP1. In other words, it is preferred according to the invention if the marker for use in determining in vitro the identity of a cartilage cell (chondrocyte) and/or a synovial cell (synoviocyte) is the ratio of ACE to MMP 1. Preferably, a value of the ratio of ACE to MMP1 above a defined threshold value indicates that the cell is a synovial cell. The threshold value is preferably less than or equal to the value of the ratio of ACE to MMP1 in a synovial cell.

With respect to further features and advantages of the marker, in order to avoid repetitions, the explanations made in the context of the above description, more particularly with respect to the first and second aspects of the invention, are also incorporated herein by reference in their entirety. The embodiments described therein also apply (mutatis mutandis) to the marker according to the sixth aspect of the invention.

According to a seventh aspect, the invention relates to the use of a cell culture, and preferably a cartilage cell culture or chondrocyte culture, for producing a medicinal product for novel therapies or an implant provided (in vitro) with cells, and preferably a tissue engineering product.

The composition or purity of the cell culture is determined by means of a marker according to the first or fifth aspect of the invention or by means of a method according to the third aspect of the invention.

The medicinal product for novel therapies or the implant preferably comprises cartilage cells or chondrocytes.

For example, the medicinal product for novel therapies can be a cell-containing two-component system, more particularly of the kit type, wherein one component comprises an albumin functionalized with thiol-reactive groups such as maleimide groups, more particularly human serum albumin, and autologous chondrocytes and the second component comprises a crosslinker functionalized with thiol groups, such as e.g. dithiopolyethylene glycol (unbranched polyethylene glycol with two terminal thiol groups). Such a medicinal product for novel therapies is distributed by the applicant under the name Novocart® Inject.

Alternatively, the medicinal product for novel therapies can be a collagen-based implant inoculated with autologous chondrocytes. In particular, the medicinal product for novel therapies can be an implant inoculated with autologous chondrocytes having a two-layer structure, in which one layer is of the membrane type, more particularly in the form of a pericardial membrane, and the second layer has a sponge-like and open-pored configuration, more particularly with columnar pores, wherein the chondrocytes are contained in the second or sponge-like layer. Such an implant is commercially distributed by the applicant under the name Novocart® 3D.

With respect to further features and advantages of the use, in order to avoid repetitions, the explanations made in the context of the above description, more particularly with respect to the first, third and fifth aspects of the invention, are also incorporated herein by reference in their entirety. The embodiments described therein also apply (mutatis mutandis) to the use according to the seventh aspect of the invention.

According to an eighth aspect, the invention relates to the use of a kit for carrying out a method according to the third or fourth aspect of the invention.

The kit comprises at least one component for determining of at least one marker selected from the group consisting of POSTN, SYNPO, MMP1, SULF1, ARHGEF28, IGF2BP1, BAIAP2L1, LIMCH1, SCIN, DCLK1, PPL, CRABP2, NES, XPNPEP2, SLIT3 and ACE.

The component can more particularly be an antibody, such as e.g. capture and/or detection antibodies, a dye such as a fluorescent dye, beads, a buffer solution, a nutrient solution, a wash solution, primers (oligonucleotides that serve as a starting point for DNA-replicating enzymes such as DNA polymerase) or a mixture/combination of at least two of the aforementioned components.

With respect to further features and advantages of the kit, more particularly of the at least one marker, in order to avoid repetitions, the explanations made in the context of the above description are also incorporated herein by reference in their entirety. The embodiments described therein also apply (mutatis mutandis) to the kit according to the eighth aspect of the invention.

Further features and advantages of the invention are found in the following description of preferred embodiments in the form of examples. The individual features can each be implemented individually or in combination with one another. The examples described below serve only to further explain the invention, and it is by no means limited thereto.

### BRIEF DESCRIPTION OF THE FIGURES

The figures show the following:

FIG. 1: scatterplot comparison of log 10 transformed synoviocyte/chondrocyte LQF intensities,

FIG. 2: volcano plot for visualization of the mean values of log 10 transformed synoviocyte/chondrocyte LFQ intensities (mean LFQ ratios),

FIG. 3: volcano plot for visualization of the differences in peptide count between synoviocytes and chondrocytes and the accompanying differences in the q value for all proteins not used in the mean rank test,

FIG. 4: mRNA expression of the synoviocyte marker ACE in chondrocytes and synoviocytes,

FIG. 5: mRNA expression of the chondrocyte marker MMP1 in chondrocytes and synoviocytes,

FIG. 6: ratio of mRNA expression of the synoviocyte marker ACE and chondrocyte marker MMP1 in chondrocytes and synoviocytes,

FIG. 7: ratio of mRNA expression of ACE/MMP1 in cell mixtures,

FIG. 8: mRNA expression of ACE in cell mixtures and

FIG. 9: mRNA expression of MMP1 in cell mixtures.

### DETAILED DESCRIPTION OF THE FIGURES

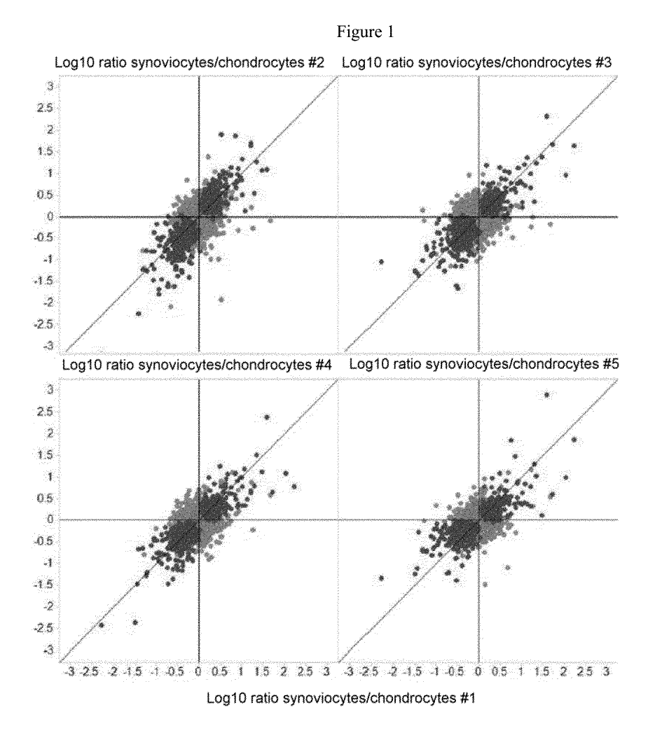

FIG. 1 shows a scatterplot comparison of the log 10 transformed synoviocyte/chondrocyte LQF intensities of donor 1 (x axis) with the other four donors. The line through the origin with slope 1 corresponds to a 100% correlation among repeated experiments. Proteins classified in the mean rank test as showing higher expression in one cell type are shown darker.

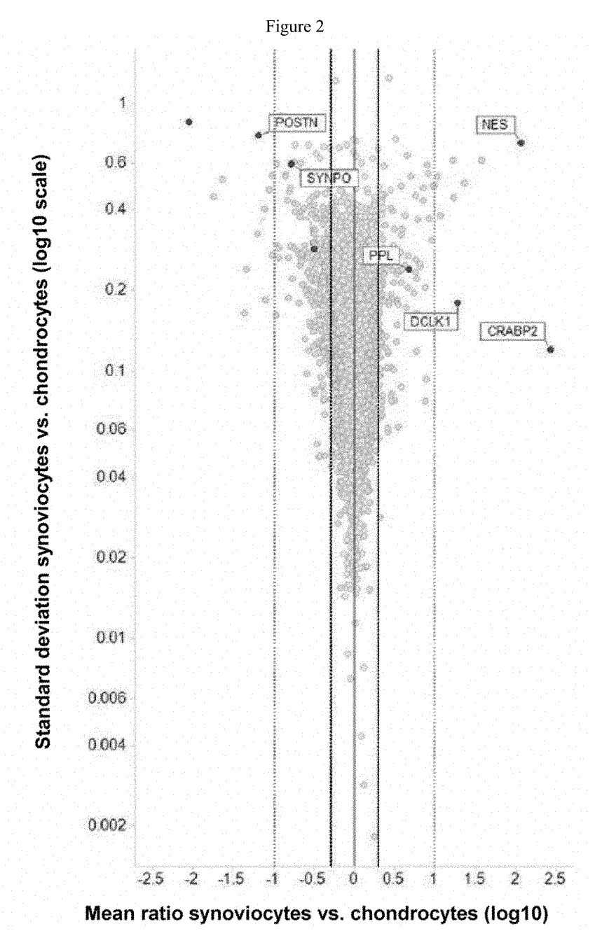

FIG. 2 shows a volcano plot for visualization of the mean values of log 10 transformed synoviocyte/chondrocyte LFQ intensities (mean LFQ ratios) against the standard deviations of the ratios of all proteins quantified in at least 3 of 5 repeated experiments. The black vertical line indicates the border between positive (=higher expression in synoviocytes) and negative (=higher expression in chondrocytes) LFQ ratios. The respective dashed lines indicate a 2- or 10-fold difference in protein expression (corresponding to ±0.3 and ±1 on the logarithmic scale).

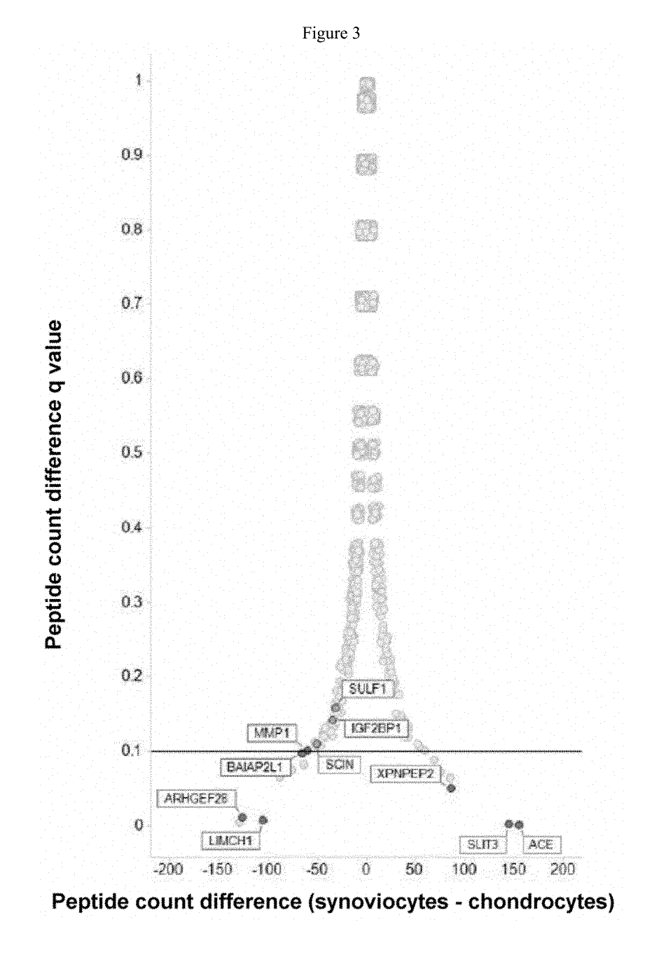

FIG. 3 shows a volcano plot for visualization of the differences in peptide count between synoviocytes and chondrocytes and the accompanying differences in q value for all proteins not used in the mean rank test. Peptide count differences greater than 1 mean higher expression in synoviocytes, and peptide count differences less than 1 mean higher expression in chondrocytes. The proteins classified in study 1 as biomarker candidates are identified by gene names.

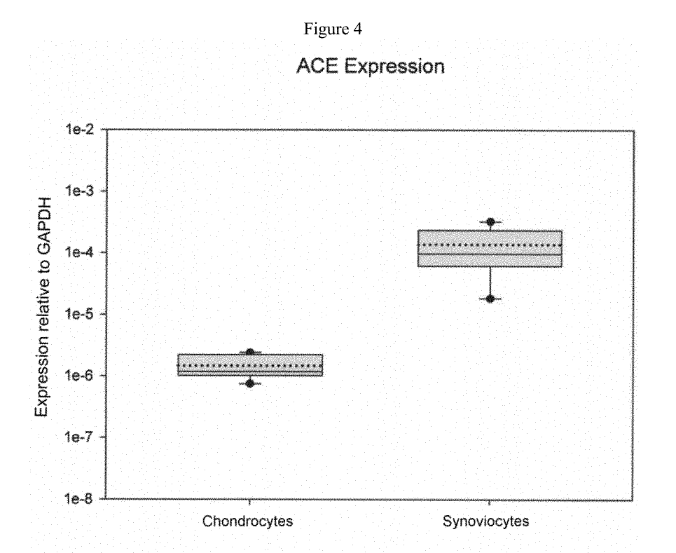

FIG. 4 shows a graph of mRNA expression of the synoviocyte marker ACE. Chondrocytes and synoviocytes from the same donor—as described below under 1. Materials and methods—were cultivated. At the end of cultivation, the mRNA expression of ACE was analyzed. The results are shown relative to expression of the reference gene GAPDH (glyceraldehyde-3-phosphate dehydrogenase) (n=10). The boxplots used in the figure show 25/75 percentiles (boxes), 10/90 percentiles (parentheses), 5/95 percentiles (points), medians (solid lines) and mean values (dotted lines). FIG. 4 shows that the mRNA expression of ACE in synoviocytes is significantly higher than in chondrocytes. ACE is therefore particularly well-suited for use as a synoviocyte marker.

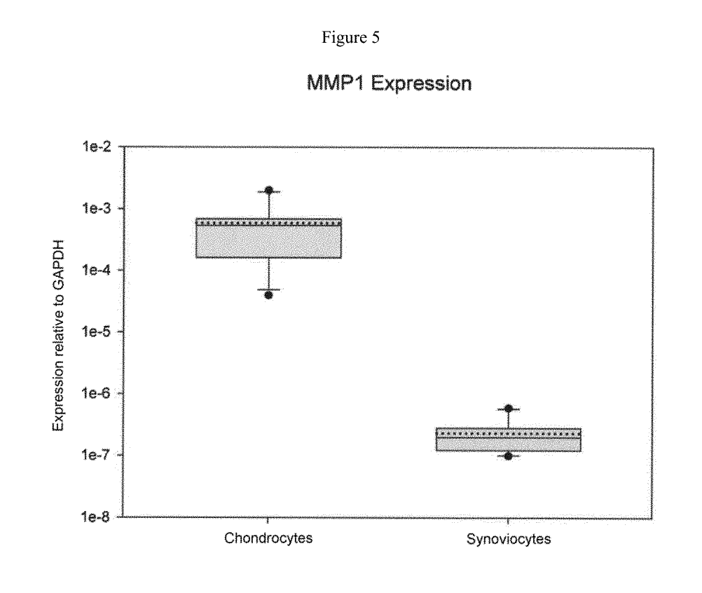

FIG. 5 shows a graph of mRNA expression of the chondrocyte marker MMP1. Chondrocytes and synoviocytes from the same donor—as described below under 1. Materials and methods—were cultivated. At the end of cultivation, the mRNA expression of MMP1 was analyzed. The results are shown relative to expression of the reference gene GAPDH (glyceraldehyde-3-phosphate dehydrogenase) (n=10). The boxplots used in the figure show 25/75 percentiles (boxes), 10/90 percentiles (parentheses), 5/95 percentiles (points), medians (solid lines) and mean values (dotted lines). FIG. 5 clearly shows that the mRNA expression of MMP1 in chondrocytes is significantly higher than in synoviocytes. MMP1 is therefore particularly well-suited for use as a chondrocyte marker.

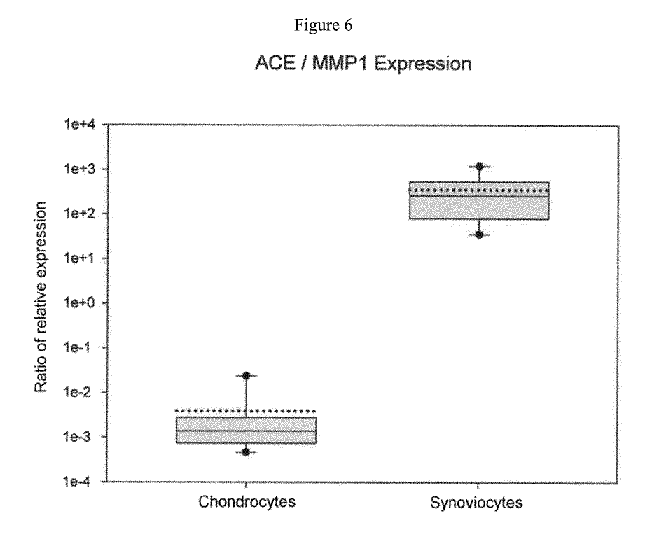

FIG. 6 shows a graph of the ratio of the expression of the synoviocyte marker ACE and the chondrocyte marker MMP1 in chondrocytes and synoviocytes (n=10). The boxplots used in the figure show the 25/75 percentiles (boxes), 10/90 percentiles (parentheses), 5/95 percentiles (points), medians (solid lines) and mean values (dotted lines). FIG. 6 shows that the ratio of ACE to MMP1 (ACE/MMP1 ratio) is also suitable as a marker within the meaning of the present invention, more particularly for determining the composition or purity of a cell culture and/or for determining in vitro the identity of a chondrocyte and/or a synoviocyte.

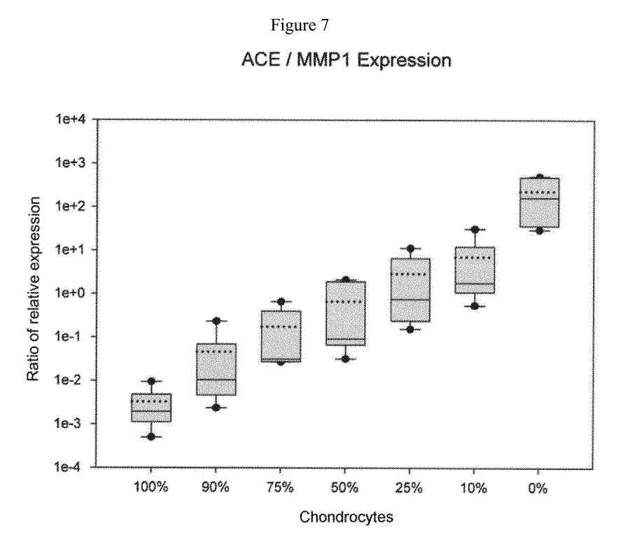

FIG. 7 shows a graph of the ratio of mRNA expression of ACE to MMP1 in cell mixtures having different contents of chondrocytes and synoviocytes (n=7). The boxplots used in the figure show the 25/75 percentiles (boxes), 10/90 percentiles (parentheses), 5/95 percentiles (points), medians (solid lines) and mean values (dotted lines). FIG. 7 shows that the value of the ratio of ACE to MMP1 correlates with the degree of purity of a chondrocyte-containing cell mixture, specifically such that a low ratio of ACE to MMP1 correlates with a high degree of purity of a chondrocyte-containing cell mixture, and a high ratio of ACE to MMP1 correlates with a low degree of purity of a chondrocyte-containing cell mixture. FIG. 7 shows in particular that the degree of purity of a chondrocyte-containing cell mixture increases with a decreasing ratio of ACE to MMP 1.

The cell mixtures were produced as follows:

Chondrocytes and synoviocytes—as described below under 1. Materials and methods—were cultivated separately from each other, and on completion of culturing, trypsinized and centrifuged. The cell pellet obtained was resuspended. The cell count of the cell suspensions was determined, and the corresponding volume of each cell suspension was then used to produce mixtures containing 100%, 90%, 75%, 50%, 25%, 10% and 0% chondrocytes. The cell mixtures prepared in this manner were again centrifuged, and the resulting cell pellet was lysed and used for gene expression analysis.

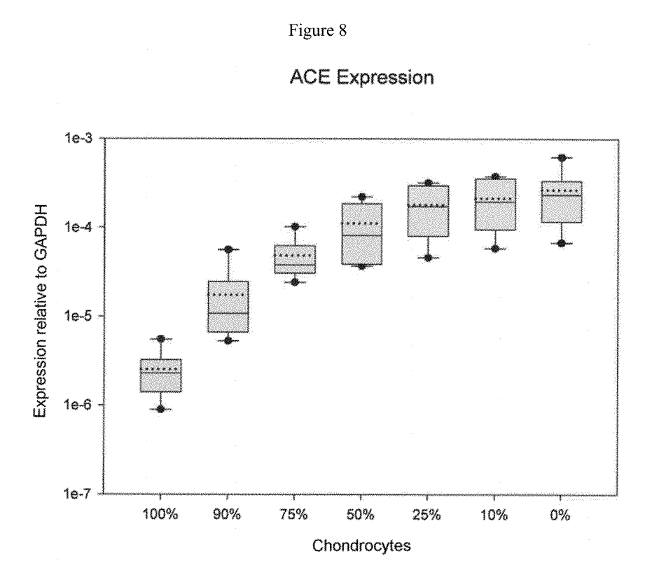

FIG. 8 shows a graph of mRNA expression of ACE in cell mixtures having different contents of chondrocytes and synoviocytes (n=7). The boxplots used in the figure show 25/75 percentiles (boxes), 10/90 percentiles (parentheses), 5/95 percentiles (points), medians (solid lines) and mean values (dotted lines).

Concerning production of the cell mixtures, reference is made to the explanations in connection with FIG. 7.

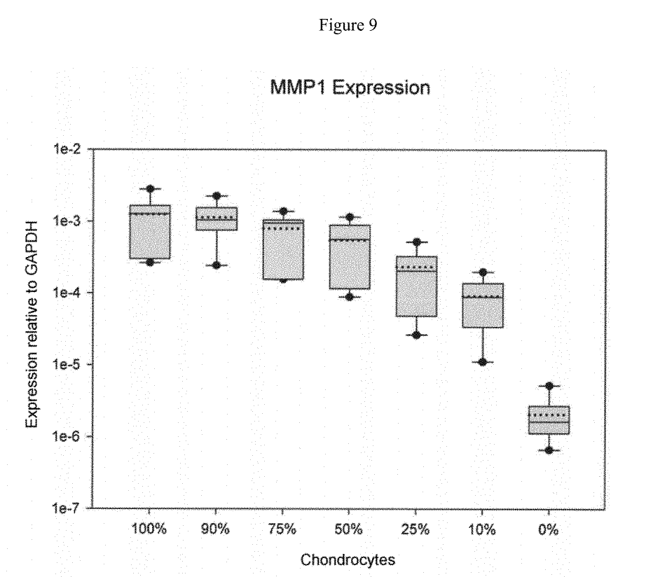

FIG. 9 shows a graph of mRNA expression of MMP1 in cell mixtures having different contents of chondrocytes and synoviocytes (n=7). The boxplots used in the figure show 25/75 percentiles (boxes), 10/90 percentiles (parentheses), 5/95 percentiles (points), medians (solid lines) and mean values (dotted lines).

Concerning production of the cell mixtures, reference is also made to the explanations in connection with FIG. 7.

### DETAILED DESCRIPTION OF THE EXEMPLARY EMBODIMENTS

### 1. Materials and Methods

### 1.1 Cell Culture, Inhibitor Treatment and Cell Lysis

The chondrocytes and synoviocytes used originated from the knee joints of deceased donors from the US. The tissue was finely chopped using two scalpels and digested with collagenase for 24 h, 5% CO₂ and 37° C. Throughout cultivation, the synovial tissue was treated identically to the cartilage tissue. After digestion, the samples were subjected to standing incubation in chondrocyte medium for 48 h, 5% CO₂ and 37° C. After this, the cells were passed through a cell sieve, centrifuged, and expanded in fresh chondrocyte medium. Depending on growth, the cells were cultivated for 19 to a maximum of 23 days, with the medium being changed 2-3 times.

After cultivation, the cells were detached from the culture surface with trypsin, washed with PBS, frozen, and sent to Evotec Munich on dry ice.

Cell lysis and enzymatic cleavage of the proteins was carried out according to a recently published protocol (Kulak, N. A., Pichler, G., Paron, I., Nagaraj, N., and Mann, M. (2014). Minimal, encapsulated proteomic-sample processing applied to copy-number estimation in eukaryotic cells. Nat Methods 11, 319-324). The cells were first stored at −80° C. and then placed on ice prior to lysis. Lysis was carried out with a cold lysis buffer (1% w/v SDC, 10 mM TCEP, 40 mM CAA, 100 mM Tris pH 8.5). Cell extracts were immediately incubated at 99° C. for 10 min and then ultrasound-treated on ice three times for 30 sec. After this, the cell debris was removed by centrifugation. The supernatant was transferred to a new reaction vessel, and protein concentration was determined (BCA Assay, Pierce). 150 μg of each cell lysate was used for subsequent enzymatic protein cleavage. A total of 10 chondrocyte and 10 synoviocyte cultures were used for analysis.

### 1.2 Sample Preparation for Mass Spectrometry (MS)

The proteins in the cell lysates were reduced with 10 MM TCEP (tris(2-carboxyethyl)phosphine) and alkylated in the presence of 40 mM CAA (2-chloroacetamide). After centrifugation, in each case, a 150 μg aliquot of the clear lysate was diluted 1:2 with H₂O, a 1:1 mixture of endopeptidase Lys-C (Wako) and trypsin (Promega) was added in an enzyme-to-protein ratio of 1:50, and incubation was carried out overnight. The resulting peptide mixture was acidified by adding 99% ethyl acetate and 1% TFA, and then desalinated using a Strata-X-C column (100 mg sorbent, Phenomenex). The peptides were eluted with 80% acetonitrile and 5% v/v ammonium hydroxide, shock-frozen in liquid nitrogen and freeze-dried. After this, the peptides were fractionated at high pH by reversed-phase chromatography (Wang, Y., Yang, F., Gritsenko, M. A., Clauss, T., Liu, T., Shen, Y., Monroe, M. E., Lopez-Ferrer, D., Reno, T., Moore, R. J., et al. (2011). Reversed-phase chromatography with multiple fraction concatenation strategy for proteome profiling of human MCF10A cells. Proteomics 11, 2019-2026). For this purpose, the peptides were reconstituted in 20 mM ammonium formiate (pH 10, buffer A), loaded onto an XBridge C18 200×4.6 mm analytical column (Waters) of an Akta Explorer System (GE Healthcare), and then separated by means of a segmented gradient with an increasing acetonitrile concentration of 7% to 30% buffer B (buffer A with 80% acetonitrile) for 15 min and then for 5 min with a gradient of up to 55%. The collected fractions of the eluted peptides were then combined in a non-linear manner such that a total of 12 samples were obtained. The samples were then desalinated with a self-produced C18 STAGEtip column (Rappsilber, J., Mann, M., and Ishihama, Y. (2007). Protocol for micro-purification, enrichment, pre-fractionation and storage of peptides for proteomics using StageTips. Nat Protoc 2, 1896-1906). For the mass spectrometric analysis, the peptides were reconstituted in 0.5% formic acid.

### 1.3 Mass Spectrometric Analysis

All LC-MS/MS analyses were conducted on a Q Exactive Plus mass spectrometer (Thermo Fisher Scientific) with the Easy n-LC 1000 UPLC System (Thermo Fisher Scientific). The samples were loaded with an autosampler onto a 40 cm quartz glass emitter (New Objective) filled with reverse phase material (Reprusil-Pur C18-AQ, 3 μm, Dr. Maisch GmbH) to a maximum pressure of 800 bar. The bound peptides were eluted in a run time of 125 min. for analysis of all fractionated samples. Peptides were directly sprayed into the mass spectrometer by means of a nano-electrospray ion source (Proxeon Biosystems). The mass spectrometer was operated in a data-dependent mode in order to allow automatic switching between total MS scans in a resolution of R=70,000 (with m/z=200) with a target value of 3,000,000 counts (max. injection time=45 ms) and MS/MS fragmentation scans at R=17,500 and a target value of 100,000 ions. The 10 most intensive peptide ions were selected by means of HCD (higher-energy collisional dissociation).

### 1.4 Data Processing

All of the raw data obtained in the tests were processed with the MaxQuant Software Suite (version 1.5.3.2) for peptide and protein identification and quantitation using the human SwissProt database (version 03 2014) (Cox, J., and Mann, M. (2008). MaxQuant enables high peptide identification rates, individualized p.p.b.-range mass accuracies and proteome-wide protein quantitation. Nat Biotechnol 26, 1367-1372; Olsen, J. V., Vermeulen, M., Santamaria, A., Kumar, C., Miller, M. L., Jensen, L. J., Gnad, F., Cox, J., Jensen, T. S., Nigg, E. A., et al. (2010). Quantitative phosphoproteomics reveals widespread full phosphorylation site occupancy during mitosis. Sci Signal 3, ra3). Label-free quantitation was activated, while the “match between runs” function was switched off. The maximum allowable mass deviation was 4.5 ppm for MS and 20 ppm for MS2 peaks. Carbamidomethylation of cysteines was input as a fixed modification, and oxidation of methionine and N-terminal acetylation were taken as variable modifications. All peptides had to have a minimum length of 7 amino acids, and a maximum of two uncleaved tryptic cleavage sites (missed cleavages) were allowed. The FDR (false discovery rate) for protein and peptide identification was set at 1%.

### 1.5 Bioinformatic Data Analysis

The protein intensities of label-free quantitation (LFQ) were used for calculating the synoviocyte/chondrocyte ratio of the same donor. This pairing resulted in a total of 5 replicates. The ratios obtained were then log 10 transformed and used for further data analysis. Proteins labeled as contaminants or reverse hits in the decoy database were removed from the modified MaxQuant protein groups table. Significant expression differences of proteins with LFQ ratios in three or more replicates were determined by the non-parametric one-sample mean rank test (Klammer M., Dybowski J. N., Hoffmann D., and Schaab C. (2014). Identification of significant features by the Global Mean Rank test. PLoS One. 2014, Aug. 13; 9(8):e104504. doi: 10.1371). The mean rank test is a global rank-based test that reliably controls the FDR (false discovery rate).

Proteins with ratios in fewer than 3 replicates were subjected to a non-parametric peptide count test, which uses the individual peptide counts for a given protein. For each protein, the mean difference in the peptide counts between chondrocytes and synoviocytes was determined. Group labels were then permuted, and the occurrence of major differences was counted over all of the proteins. This was repeated for all possible permutations using the average number of false discoveries in order to estimate the false discovery (FD) of a given protein.

For combined analysis of the two studies, a total of 10 donors with related chondrocytes and synoviocytes were used. Significant expression differences in proteins with LFQ ratios in at least 7 replicates were determined by the mean rank test, and all other proteins were subjected to the non-parametric peptide count test.

### 1.6 Comparison of the Two Data Sets:

In order to allow comparison of the results of the two individual studies with 5 donors each, the data were compared by either the UniProtKB identifiers and/or the gene name. Based on the quality of the comparison, the data were divided into the following four categories: (++) identical UniProtKB identifiers; (+) partially identical UniProtKB identifiers and identical gene names; (+/−) partially identical UniProtKB identifiers and partially matching gene name; (−) not applicable.

### 1.7 Gene Expression Analysis

On completion of cultivation, an aliquot of 0.5 million was taken from the removed cells for analysis of gene expression, centrifuged, and the cell pellet was lysed with lysis buffer. Subsequent RNA isolation was carried out using an RNeasy mini kit 250 (Qiagen). cDNA synthesis was carried out with the Advantage® RT for PCR kit (Clontech).

Gene-specific primer sequences were designed at the NCBI with PrimerBlast and synthesized by Biotez. The primers were used in a concentration of 100 nM or 200 nM/well, and expression of mRNA was analyzed by the real-time PCR method on the Roche LC480. The fluorescent dye SYBR Green was used for detection of the amplified sequences.

### 2. Results:

In total, with a determined FDR of <1%, 10,702 different proteins were identified. A similar number of proteins were identified for chondrocytes and synoviocytes (10,550 or 10,462). The identifications were based on more than 2,300,000 peptide detections and 162,299 identified peptides. Throughout all of the experiments, a comparable number of proteins was identified, which speaks for the basic robustness of the workflow used for proteomic analysis.

In order to show the reproducibility of protein quantitation over biological replicates of 5 donors with related chondrocyte and synoviocyte preparations, scatterplot analyses of label-free quantitation ratios (LFQ ratios) were carried out. For this purpose, the intensities of the LFQ were converted into log 10 transformed ratios that represent the ratio of the LFQ intensity of the synoviocytes to the LFQ intensity of the chondrocytes.

As an example, the results for donor 1 are shown in FIG. 1. In addition to a large cloud of log 10 transformed protein ratios around zero (=around a linear ratio of approximately 1), which means that the proteins are expressed identically in both cell types, one finds a few proteins that show a different frequency in the two cell types. The scatterplot comparisons show a high correlation of the donors, as the great majority of the proteins are on a line through the origin with slope 1. The results obtained were comparable for the two studies.

In the next step, further data analyses were conducted in order to identify proteins with significantly different expression levels between the two cell types. For this purpose, all of the proteins quantitated in at least 3 of the 5 biological replicates were subjected to a statistical analysis. These requirements were met by a total of 7,361 proteins in study 2 and 5,378 proteins in study 1. Significant and reproducible changes were identified using the mean rank test with an FDR of 10% (q values ≤0.1) (Klammer M., Dybowski J. N., Hoffmann D., and Schaab C. (2014). Identification of significant features by the Global Mean Rank test. PLoS One. 2014, Aug. 13; 9(8):e104504. doi: 10.1371). The FDR of 10% used is an arbitrarily selected value that appeared to be suitable for the further bioinformatic analyses of the study, such as gene ontology enrichment and interaction network analysis. The distribution of the proteins with significantly differing protein expression between the two cell types can be visualized using so-called volcano plots. For this purpose, the mean values of the log 10 transformed synoviocyte/chondrocyte LFQ intensities (mean LFQ ratio) were plotted against the standard deviation of the ratios of all proteins. All of the proteins with differential expression between the two cell types are shown in red. Proteins classified as biomarker candidates were labeled by name.

After application of the mean rank test, approx. 15% of all proteins analyzed in the test were found to differ significantly in chondrocytes and synoviocytes (total of 1,135). In the next step, a peptide count was carried out in order to identify marker proteins that were expressed as exclusively as possible in only one of the two cell types. This test compares the average number of peptides by means of which one protein was identified in the two cell types. For this test, a total of 3,340 proteins were used, 16 of which were differentially expressed (10% FDR, q values ≤0.1). Of these 16 proteins, 8 were expressed in only one type or expressed to a significantly greater degree in only one cell type.

Selected proteins with specific protein expression in chondrocytes or synoviocytes that were found by means of the peptide count test in both studies are shown in Table 1.

TABLE 1
Selected proteins with differential expression in chondrocytes or synoviocytes determined by means of
the peptide test. A: results of the first study, B: results of the second study.
A

| ID | | | | | | | |
|---|---|---|---|---|---|---|---|
| (EVT03802) | Protein name | Gene name | Cho. #1 | Cho. #2 | Cho. #3 | Cho. #4 | Cho. #5 |
| 9320 | Adseverin | SCIN | 23 | 17 | 4 | 10 | 3 |
| 1669 | Collagen alpha-1(XI) chain | COL11A1 | 10 | 19 | 13 | 19 | 9 |
| 1317 | Interstitial collagenase | MMP1 | 18 | 11 | 13 | 4 | 2 |
| 398 | Laminin subunit alpha-5 | LAMA5 | 2 | 4 | 9 | 34 | 0 |
| 2995 | Lysyl oxidase homolog 3 | LOXL3 | 24 | 26 | 14 | 22 | 7 |
| 1687 | Anglotensin-converting enzyme | ACE | 0 | 0 | 0 | 0 | 0 |
| 6504 | Dedicator of cytokinesis protein 10 | DOCX10 | 0 | 1 | 5 | 2 | 0 |
| 6968 | Peroxisomal acyl-coenzyme A oxidase 2 | ACOX2 | 0 | 0 | 0 | 0 | 0 |
| 5228 | Rap1-GTP-interacting Adaptor Molecule* | AP881IP | 0 | 0 | 0 | 2 | 1 |
| 735 | Slit homolog 3 protein | SLIT3 | 0 | 0 | 0 | 2 | 0 |
| 589 | Xaa-Pro aminopeptidase 2 | XPNPEP2 | 1 | 0 | 0 | 0 | 0 |

| ID | | | | | | Peptide Count |
|---|---|---|---|---|---|---|
| (EVT03802) | Syn #1 | Syn #2 | Syn #3 | Syn #4 | Syn #5 | q-value |
| 9320 | 0 | 0 | 0 | 0 | 1 | 0.068 |
| 1669 | 1 | 1 | 0 | 2 | 3 | 0.068 |
| 1317 | 1 | 0 | 0 | 0 | 0 | 0.068 |
| 398 | 0 | 0 | 0 | 0 | 0 | 0.069 |
| 2995 | 1 | 1 | 1 | 2 | 1 | 0.062 |
| 1687 | 10 | 7 | 8 | 13 | 10 | 0.069 |
| 6504 | 15 | 12 | 14 | 13 | 13 | 0.068 |
| 6968 | 6 | 8 | 5 | 9 | 10 | 0.098 |
| 5228 | 22 | 17 | 16 | 14 | 15 | 0.062 |
| 735 | 14 | 18 | 16 | 25 | 24 | 0.062 |
| 589 | 16 | 7 | 7 | 6 | 5 | 0.098 |

B

| ID | | | | | | | |
|---|---|---|---|---|---|---|---|
| (EVT03867) | Protein name | Gene name | Cho. #1 | Cho. #2 | Cho. #3 | Cho. #4 | Cho. #5 |
| 9320 | Adseverin | SCIN | 9 | 18 | 12 | 8 | 5 |
| 1669 | Collagen alpha-1(XI) chain | COL11A1 | 24 | 36 | 33 | 38 | 16 |
| 1317 | Interstitial collagenase | MMP1 | 14 | 6 | 12 | 12 | 13 |
| 398 | Laminin subunit alpha-5 | LAMA5 | 26 | 13 | 3 | 2 | 1 |
| 2995 | Lysyl oxidase homolog 3 | LOXL3 | 23 | 15 | 17 | 18 | 17 |
| 1687 | Anglotensin-converting enzyme | ACE | 3 | 0 | 0 | 1 | 0 |
| 6504 | Dedicator of cytokinesis protein 10 | DOCX10 | 14 | 7 | 10 | 3 | 2 |
| 2604 | Nestin | NES | 21 | 32 | 9 | 12 | 5 |
| 6988 | Peroxisomal acyl-coenzyme A oxidase 2 | ACOX2 | 0 | 1 | 0 | 1 | 0 |
| 5228 | Rap1-GTP-interacting Adaptor Molecule* | AP881IP | 5 | 0 | 0 | 0 | 5 |
| 735 | Slit homolog 3 protein | SLIT3 | 5 | 0 | 0 | 0 | 0 |
| 589 | Xaa-Pro aminopeptidase 2 | XPNPEP2 | 0 | 0 | 0 | 0 | 1 |

| ID | | | | | | Peptide Count | Mean Rank | |
|---|---|---|---|---|---|---|---|---|
| (EVT03867) | Syn #1 | Syn #2 | Syn #3 | Syn #4 | Syn #5 | q-value | q-value | Match type |
| 9320 | 0 | 0 | 0 | 0 | 0 | 0.107 | — | + |
| 1669 | 2 | 1 | 0 | 13 | 0 | 0.000 | — | ++ |
| 1317 | 0 | 0 | 0 | 0 | 0 | 0.100 | — | ++ |
| 398 | 0 | 0 | 2 | 0 | 2 | 0.127 | — | ++ |
| 2995 | 2 | 1 | 5 | 0 | 6 | — | 0.000 | ++ |
| 1687 | 33 | 40 | 33 | 31 | 23 | 0.000 | — | ++ |
| 6504 | 33 | 21 | 20 | 21 | 21 | — | 0.008 | ++ |
| 2604 | 61 | 60 | 49 | 57 | 49 | — | 0.000 | ++ |
| 6988 | 17 | 21 | 12 | 21 | 15 | 0.065 | — | ++ |
| 5228 | 18 | 19 | 15 | 14 | 19 | 0.050 | — | ++ |
| 735 | 37 | 25 | 32 | 29 | 27 | 0.000 | — | ++ |
| 589 | 22 | 20 | 16 | 20 | 11 | 0.060 | — | ++ |

*(allas) Amyloid beta AA precursor protein-binding family B member 2-interacting protein

Proteins determined by means of bioinformatic analysis to be expressed in a significantly differing manner are shown in Table 2.

TABLE 2
Markers for chondrocytes and synoviocytes.

| | ID | ID | | | | | | | |
|---|---|---|---|---|---|---|---|---|---|
| | (Protein | (Protein | | | | | | | |
| | group, | group, | | | Match | Test used | q-value | Test used | q-value |
| Cell type | EVT03802) | EVT03867) | Protein name | Gene name | type | (EVT03802) | (EVT03802) | (EVT03867) | (EVT03867) |
| Chondrocytes | 4057 | 4524 | Periostin | POSTN | ++ | Mean Rank | 0.009 | Mean Rank | 0.009 |
| | 5679 | 6480 | Synaptopodin | SYNPO | + | Peptide Count | 0.068 | Mean Rank | 0.023 |
| | 1317 | 1522 | Interstitial | MMP1 | ++ | Peptide Count | 0.069 | Peptide Count | 0.100 |
| | | | collagenase | | | | | | |
| | 5512 | 6273 | Extracellular | SULF1 | ++ | Mean Rank | 0.009 | Peptide Count | 0.162 |
| | | | sulfatase Sulf-1 | | | | | | |
| | 5645 | 6432 | Rho guanine | ARHGEF28 | + | Peptide Count | 0.098 | Peptide Count | 0.003 |
| | | | nucleotide | | | | | | |
| | | | exchange factor | | | | | | |
| | | | 28 | | | | | | |
| | 8360 | 9665 | Insulin-like | IGF2BP1 | + | Peptide Count | 0.098 | Peptide Count | 0.134 |
| | | | growth factor-2 | | | | | | |
| | | | mRNA-binding | | | | | | |
| | | | protein 1 | | | | | | |
| | 8638 | 9976 | Brain-specific | BAIAP2L1 | ++ | Peptide Count | 0.098 | Peptide Count | 0.091 |
| | | | angiogenesis | | | | | | |
| | | | inhibitor 1- | | | | | | |
| | | | associated | | | | | | |
| | | | protein 2-like | | | | | | |
| | | | protein 1 | | | | | | |
| | 8901 | 10270 | LIM and calponin | LIMCH1 | + | Peptide Count | 0.068 | Peptide Count | 0.014 |
| | | | homology | | | | | | |
| | | | domains- | | | | | | |
| | | | containing | | | | | | |
| | | | protein 1 | | | | | | |
| | 9320 | 10748 | Adseverin | SCIN | + | Peptide Count | 0.069 | Peptide Count | 0.107 |
| Synoviocytes | 367 | 440 | Serine/threonine- | DCLK1 | + | Mean Rank | 0.000 | Mean Rank | 0.000 |
| | | | protein kinase | | | | | | |
| | | | DCLK1 | | | | | | |
| | 640 | 763 | Periplakin | PPL | + | Mean Rank | 0.000 | Mean Rank | 0.004 |
| | 2164 | 2455 | Cellular retinoic | CRABP2 | ++ | Mean Rank | 0.000 | Mean Rank | 0.000 |
| | | | acid- binding | | | | | | |
| | | | protein 2 | | | | | | |
| | 2604 | 2927 | Nestin | NES | ++ | Peptide Count | 0.000 | Mean Rank | 0.000 |
| | 589 | 703 | Xaa-Pro | XPNPEP2 | ++ | Peptide Count | 0.098 | Peptide Count | 0.000 |
| | | | aminopeptidase 2 | | | | | | |
| | 735 | 876 | Slit homolog 3 | SLIT3 | ++ | Peptide Count | 0.062 | Peptide Count | 0.000 |
| | | | protein | | | | | | |
| | 1687 | 1928 | Angiotensin- | ACE | ++ | Peptide Count | 0.069 | Peptide Count | 0.000 |
| | | | converting | | | | | | |
| | | | enzyme | | | | | | |

In addition, for the proteins classified as biomarker candidates, the difference in protein expression between the two cell types (-fold difference) and the mean peptide counts are summarized below (Table 3).

TABLE 3
Differences in expression of the markers between chondrocytes and synoviocytes and mean
value of peptide counts (shown separately according to study 1 and study 2 respectively).

| | | | | EVT3802_Fold | EVT3867_Fold |
|---|---|---|---|---|---|
| | | | | difference in | difference in |
| | | Test used | Test used | protein | protein |
| Cell type | Gene name | (EVT03802) | (EVT03867) | expression | expression |
| Chondrocytes | POSTN | Mean Rank | Mean Rank | 46.7 | 15.2 |
| | SYNPO | Peptide Count | Mean Rank | 17.3 | 6.0 |
| | MMP1 | Peptide Count | Peptide Count | | |
| | SULF1 | Mean Rank | Peptide Count | 46.1 | 2.8 |
| | ARHGEF28 | Peptide Count | Peptide Count | | |
| | IGF2BP1 | Peptide Count | Peptide Count | | |
| | BAIAP2L1 | Peptide Count | Peptide Count | 3.7 | 7.5 |
| | LIMCH1 | Peptide Count | Peptide Count | | 6.7 |
| | SCIN | Peptide Count | Peptide Count | | |
| Synoviocytes | DCLK1 | Mean Rank | Mean Rank | 67.7 | 19.0 |
| | PPL | Mean Rank | Mean Rank | 116.5 | 4.7 |
| | CRABP | Mean Rank | Mean Rank | 71.7 | 269.8 |
| | NES | Peptide Count | Mean Rank | 26.6 | 114.3 |
| | XPNPEP2 | Peptide Count | Peptide Count | | |
| | SLIT3 | Peptide Count | Peptide Count | 23.2 | 81.4 |
| | ACE | Peptide Count | Peptide Count | | [symbol] |

| | | EVT3802_Mean | EVT3802_Mean | EVT3867_Mean | EVT3867_Mean |
|---|---|---|---|---|---|
| | | value peptide | value peptide | value peptide | value peptide |
| | | count, | count, | count, | count, |
| | | synoviocytes | chondrocytes | synoviocytes | chondrocytes |
| Cell type | Gene name | (#1-#5) | (#1-#5) | (#1-#5) | (#1-#5) |
| Chondrocytes | POSTN | 7 | 54.6 | 12.4 | 30.6 |
| | SYNPO | 0.6 | 13.2 | 4.6 | 17 |
| | MMP1 | 0.2 | 9.6 | 0 | 11.2 |
| | SULF1 | 1.8 | 17.8 | 3.2 | 9.2 |
| | ARHGEF28 | 0 | 7.8 | 0.8 | 25.4 |
| | IGF2BP1 | 0 | 7.6 | 0.6 | 7.8 |
| | BAIAP2L1 | 0.6 | 8.4 | 1.4 | 14.2 |
| | LIMCH1 | 0.2 | 12.8 | 1.2 | 22.6 |
| | SCIN | 0.2 | 10.4 | 0 | 10.4 |
| Synoviocytes | DCLK1 | 29.8 | 2.6 | 23.2 | 10.6 |
| | PPL | 131.6 | 10.8 | 89.6 | 56.6 |
| | CRABP | 14.8 | 2.8 | 8 | 2 |
| | NES | 60.6 | 5.8 | 55.2 | 15.8 |
| | XPNPEP2 | 8.2 | 0.2 | 17.8 | 0.2 |
| | SLIT3 | 19.4 | 0.4 | 30 | 1.2 |
| | ACE | [symbol] | 0 | [symbol] | 0.8 |

[symbol]

indicates data missing or illegible when filed

1.-15. (canceled)

16. A method for assessing the composition or purity of a cell culture, comprising the following steps:
a) obtaining a cell sample from a cell culture,
b) determining the expression level of at least one marker in the cell sample and
c) determining the composition or purity of the cell culture based on the expression level of the at least one marker,
characterized in that the at least one marker is selected from the group consisting of ACE, MMP1, POSTN, SYNPO, SULF1, ARHGEF28, IGF2BP1, BAIAP2L1, LIMCH1, SCIN, DCLK1, PPL, CRABP2, NES, XPNPEP2, SLIT3 and combinations of at least two of the aforementioned markers.

17. The method of claim 16, wherein the expression level of the at least one marker is determined in a cell sample obtained from the cell culture.

18. The method of claim 16, wherein the cell culture is a chondrocyte culture.

19. The method of claim 16, wherein the cell culture contains cells that originate from a cartilage biopsy specimen, preferably a cartilage/bone punch biopsy specimen, more particularly a cartilage tissue fragment thereof.

20. The method of claim 19, wherein the biopsy specimen is a biopsy specimen from a joint, more particularly a knee joint.

21. The method of claim 16, wherein the at least one marker is selected from the group consisting of MMP1, POSTN, SYNPO, SULF1, ARHGEF28, IGF2BP1, BAIAP2L1, LIMCH1, SCIN and combinations of at least two of the aforementioned markers, wherein an expression level of the at least one marker above a defined threshold level indicates that the cell culture contains chondrocytes.

22. The method of claim 21, wherein the threshold value is greater than or equal to the expression level of the at least one marker in a pure synovial cell culture.

23. The method of claim 16, wherein the at least one marker is selected from the group consisting of ACE, DCLK1, PPL, CRABP2, NES, XPNPEP2, SLIT3 and combinations of at least two of the aforementioned markers, wherein an expression level of the at least one marker above a defined threshold level indicates that the cell culture contains synovial cells.

24. The method of claim 23, wherein the threshold value is greater than or equal to the expression level of the at least one marker in a pure chondrocyte culture.

25. The method of claim 16, wherein the at least one marker is the ratio of ACE to MMP1 or the inverse ratio of the two.

26. The method of claim 25, wherein the value of the ratio of ACE to MMP1 above a defined threshold level indicates that the cell culture contains synovial cells, wherein the threshold value is preferably less than or equal to the value of the ratio of ACE to MMP1 in a pure synovial cell culture.

27. The method of claim 16, wherein the expression level is determined on an RNA basis, preferably an mRNA basis, or a protein basis.

28. The method of claim 16, comprising the step of using a kit, wherein the kit comprises at least one component for detection of at least one marker selected from the group consisting of ACE, MMP1, POSTN, SYNPO, SULF1, ARHGEF28, IGF2BP1, BAIAP2L1, LIMCH1, SCIN, DCLK1, PPL, CRABP2, NES, XPNPEP2 and SLIT3.

29. A method for producing a medicinal product for novel therapies, more particularly a tissue engineering product, comprising the step of using a cell culture, wherein the composition or purity of the cell culture is determined by means of at least one marker selected from the group consisting of ACE, MMP1, POSTN, SYNPO, SULF1, ARHGEF28, IGF2BP1, BAIAP2L1, LIMCH1, SCIN, DCLK1, PPL, CRABP2, NES, XPNPEP2 and SLIT3.
